SEQUENCE LISTING (3 sequences)

SEQ ID NO: 1 - protein, 11 aa, Artificial Sequence
  Synthetically generated peptide
YGRKKRRQRRR

SEQ ID NO: 2 - protein, 11 aa, Artificial Sequence
  Synthetically generated peptide
YGKKKKKQKKK

SEQ ID NO: 3 - RNA, 29 nt, Human Immunodeficiency Virus
ggcagaucugagccugggagcucucugcc

(19) United States
(12) Patent Application Publication
(10) Pub. No.: US 2005/0064470 A1
(43) Pub. Date: Mar. 24, 2005

(54) High throughput screening methods for identifying RNA binding compounds

(76) Inventor: Tariq M. Rana, Shrewsbury, MA (US)

Correspondence Address: FISH & RICHARDSON PC, 225 FRANKLIN ST, BOSTON, MA, 02110, US

(21) Appl. No.: 10/893,722

(22) Filed: Jul. 16, 2004

Related U.S. Application Data
(60) Provisional application No. 60/488,041, filed on Jul. 17, 2003.
(60) Provisional application No. 60/493,229, filed on Aug. 6, 2003.

(51) Int. Cl. C12Q 1/68

(52) U.S. Cl. 435/6

(57) ABSTRACT

The present invention provides methods for high-throughput screening of combinatorial libraries for specific RNA-binding compounds, using fluorescence polarization anisotropy.

### CLAIM OF PRIORITY

This application claims priority under 35 USC §119(e) from U.S. Provisional Patent Application Nos. 60/488,041, filed on Jul. 17, 2003, and 60/493,229, filed on Aug. 6, 2003, the entire contents of which are incorporated herein by reference.

### FEDERALLY SPONSORED RESEARCH OR DEVELOPMENT

This invention was made with Government support under Grant Nos. AI45466, AI43198, and A141404, awarded by the National Institutes of Health. The Government has certain rights in the invention.

### TECHNICAL FIELD

This invention relates to assays for RNA binding molecules using fluorescence polarization anisotropy, and more particularly to high throughput assays for the same.

### BACKGROUND

Nucleic acids, and in particular ribonucleic acids (RNAs), are capable of folding into complex secondary and tertiary structures that include bulges, loops, turns, and pseudoknots (Chastain et al., Progress in Nucleic Acid Res. Mol. Biol. 41:131-177 (1991); Chow et al., Chemical Reviews 97:1489-1514 (1997), which can provide binding sites for in vivo ligands, such as proteins and other RNAs. When they occur in RNAs that interact with proteins, these structures are found to play important roles in protein-RNA interactions. A particular protein-binding RNA structure can be a molecular receptor not only for the protein with which it interacts, but also for synthetic compounds, which may be antagonists of the protein-RNA interaction. Since RNA-protein and RNA-RNA interactions are important in a variety of cellular functions, including transcription, RNA splicing, RNA stability, and translation, and can be important in a variety of disorders, including viral and microbial disease progression, it would be advantageous to have a general method for rapidly identifying synthetic compounds for targeting specific RNA structures.

Synthetic molecules that can bind with high affinity to specific sequences of single- or double-stranded nucleic acids have the potential to interfere with these interactions in a controllable way, making them attractive tools for molecular biology and medicine. The diversity of secondary and tertiary structure, however, makes it difficult to rationally design synthetic agents with general, simple-to-use recognition rules analogous to those for the formation of double- and triple-helical nucleic acids.

### SUMMARY

This invention is based, in part, on the discovery that, by using an optimized fluorescent polarization anisotropy assay (FPA) together with small RNAs having specific, well-defined structures, one can identify small molecule RNA binding agents. Thus, one can create high throughput screening methods for identifying small molecules that bind to RNA for the purpose of specifically disrupting RNA-protein interactions. The methods can be used to screen combinatorial libraries, e.g., small molecule libraries. The compounds to be screened and the target RNA can be free in solution or either one can be attached to a solid substrate. The new methods are useful for identifying small molecule drug leads for targeting specific RNA-protein interactions.

Thus, the invention provides methods, e.g., high-throughput methods, for simultaneously screening a plurality of test compounds, e.g., at least five test compounds, for their ability to bind to a target RNA molecule. The methods include incubating the plurality of test compounds with a target RNA molecule having a fluorescent label, under conditions that enable the binding of the test compounds to the target RNA molecule; and assaying fluorescence polarization of the target RNA molecule. An increase, e.g., a statistically significant increase, in fluorescence polarization of the target RNA molecule, as compared to a level of fluorescence polarization of the target RNA molecule in the absence of any test compounds, indicates that one of the test compounds binds to the target RNA molecule.

In some embodiments, the methods include incubating the test compounds and target RNA in a housing having a plurality of individual areas, e.g., a multiwell plate, with each area including a target RNA and one or more of the plurality of test compounds.

The invention also provides methods for simultaneously screening a plurality of test compounds, e.g., a viral, bacterial, fungal, or parasitic infection, to identify a candidate compound for the treatment of a pathogen-associated condition. The method includes incubating a plurality of test compounds with a target RNA molecule derived from the pathogen, wherein the target RNA has a fluorescent label, under conditions that enable the binding of the test compound to the target RNA molecule; and assaying fluorescence polarization of the target RNA molecule. An increase, e.g., a statistically significant increase, in the fluorescence polarization of the target RNA molecule, as compared to a level of fluorescence polarization of the target RNA molecule in the absence of any test compounds, indicates that one of the test compounds is a candidate compound for the treatment of the pathogen-associated condition. As used herein, a “target RNA molecule derived from a pathogen” is a target RNA having a sequence that is identical to a region of the sequence of an RNA endogenous to the pathogen.

In some embodiments, the pathogen-associated condition is a viral infection and the target RNA molecule is derived from viral RNA, e.g., Human Immunodeficiency Virus (HIV), e.g., trans-activation response element (TAR) and Rev response element (RRE) RNA; Hepatitis C Virus (HCV); and Severe Acute Respiratory Syndrome (SARS). As used herein, a “target RNA molecule derived from viral RNA” is a target RNA having a sequence that is identical to a region of the sequence of a natural viral RNA.

In some embodiments, the pathogen-associated condition is a bacterial infection and the target RNA molecule is derived from RNA of the bacterium associated with the condition, e.g., bacterial rRNA. In some embodiments, the bacterial RNA is from Escherichia coli, Mycobacterium tuberculosis, Neisseria gonorrhoeae, Neisseria meningitidis, Rickettsiae, Coxiella burnetii, Salmonellae, Staphylococcus aureus, Streptococcus pyogenes, or Treponema pallidum. As used herein, a target RNA molecule derived from bacterial RNA is a target RNA having a sequence that is identical to a region of the sequence of a natural bacterial RNA.

In some embodiments, the methods described herein include administering a candidate compound to an animal model of the pathogen-associated condition. An improvement in a symptom of the animal model, e.g., a clinical symptom, indicates that the candidate compound is a candidate therapeutic compound for the treatment of the pathogen-associated condition.

In some embodiments, the method includes administering the candidate therapeutic compound to a subject, e.g., a human in a clinical trial, having the pathogen-associated condition, wherein an improvement in the subject indicates that the compound is a therapeutic agent.

In some embodiments, the pathogen-associated condition is a bacterial infection, and the method further includes contacting a candidate compound with the bacterium associated with the condition, and evaluating an effect of the candidate compound on the viability of the bacterium. A reduction in the viability of the bacterium (e.g., increased death or reduced growth rates) indicates that the compound is a candidate therapeutic compound for the treatment of the bacterial infection.

In one aspect, the invention features methods of screening a plurality of small molecules for their ability to bind to a target RNA molecule, e.g., a target RNA molecule having at least one secondary structure. The methods include providing one or more pre-selected target RNA molecules labeled with a fluorescent label; providing a plurality of small molecules; and detecting the fluorescence polarization of the target RNA molecules in the presence and absence of at least two, e.g., at least three, four, five, six, or more, of the plurality of small molecules. An increase in the fluorescence polarization of the target RNA molecules caused by a particular small molecule indicates that the particular small molecule binds to the target RNA molecule. In an alternative method, the target RNA can be unlabeled, and the small molecules can be labeled with a fluorescent dye. The fluorescent label can be any fluorochrome, so long as the biological activity of the small molecule is unperturbed by the dye modification. A number of suitable fluorescent labels are known in the art, including, e.g., fluorescein, eosin, BODIPY™ dyes (dibenzopyrrometheneboron difluoride dyes, based on 4,4-difluoro-4-bora-3a,4a-diaza-s-indacene dyes, available from Molecular Probes Eugene, Oreg.), ALEXA FLUOR™ dyes (available from Molecular Probes Eugene, Oreg.), fluorescein, Oregon Green, tetramethylrhodamine and Texas Red. Many are commercially available, e.g., from Molecular Probes (Eugene, Oreg.).

In some embodiments, the target RNA has one or more secondary structure, e.g., one or more hairpins, internal loops, stacked pairs, multi-branch loops, external bases, and bulges. The target RNA molecule can be, e.g., less than or equal to about 100, 75, 50, 35, 30, 25, or 20 bases long. The target RNA molecule can be derived from the sequence of any RNA of interest, e.g., any nuclear RNA, microRNA (mRNA), mRNA, ribosomal RNA, or regulatory RNA. In some embodiments, the target RNA is derived from TAR. The target RNA molecule can be derived from mammalian RNA, e.g., human RNA, bacterial RNA, viral RNA, e.g., HIV (e.g., TAR or RRE), HCV, or SARS, or RNA from other pathogens. In some embodiments, the target RNA is derived from a regulatory RNA, e.g., an RNA that affects the posttranscriptional regulation of gene expression or affects the stability of an mRNA.

As used herein, “small molecules” refers to small organic or inorganic molecules of molecular weight below about 3,000 Daltons. In some embodiments, the small molecules are peptide molecules; peptidomimetics (e.g., peptoid oligomers, e.g., peptoid amide or ester analogues, β-peptides, D-peptides, L-peptides, oligourea or oligocarbamate); small peptides (e.g., tripeptides, tetrapeptides, pentapeptides, hexapeptides, heptapeptides, octapeptides, nonapeptides, decapeptides, or larger, e.g., 20-mers or more); cyclic peptides; other non-natural or unnatural peptide-like structures; and inorganic molecules (e.g., heterocyclic ring molecules). In some embodiments, the small molecules are nucleic acids. In some embodiments, the plurality of small molecules includes at least 48 96, 128, 256, or 384 different small molecules, e.g., each of the small molecules differs from all of the other small molecules. In some embodiments, each of the plurality of small molecules is bound to a solid support, e.g., a bead.

In some embodiments, the small molecules are free in solution. In some embodiments, the detecting step includes detecting the fluorescence polarization of the target RNA molecules in the presence and absence of each one of the plurality of small molecules. Detecting the fluorescence polarization of the target RNA molecules in the presence and absence of at least two, three, four, five or more of the plurality of small molecules can be performed simultaneously, e.g., in the same well, or in different wells.

In another aspect, the invention includes small molecules identified as capable of binding to a target RNA molecule by a method described herein. A test compound that has been screened by a method described herein and determined to bind to a target RNA molecule, can be considered a candidate compound. A candidate compound that has been screened, e.g., in an in vivo model of a disorder, e.g., inflammation, viral infection, or cancer, and determined to have a desirable effect on the disorder, e.g., on one or more symptoms of the disorder, can be considered a candidate therapeutic agent. Candidate therapeutic agents, once screened in a clinical setting, are therapeutic agents, which can then be optionally optimized and/or derivatized, and formulated with physiologically acceptable excipients to form pharmaceutical compositions. In some embodiments, the test compound, candidate compound, or candidate therapeutic compound is a β-peptide, e.g., a β-peptide including the sequence of SEQ ID NO:1, and is useful, e.g., in the treatment of HIV. The invention also includes pharmaceutical compositions including the small molecules, and optionally a pharmaceutically acceptable vehicle.

The invention further provides methods of treating, preventing, or delaying the development or progression of a disorder in a subject, e.g., inflammation, viral infection, or cancer, by administering to the patient a therapeutically effective amount of a pharmaceutical composition described herein. The methods can include administering a therapeutically effective amount of a pharmaceutical composition including β-peptide of SEQ ID NO:1 to a subject identified as having HIV.

As used herein, “RNA secondary structure” refers to a structure formed by a single-stranded RNA folding back upon itself in solution. Secondary structures can include duplexes, single-stranded regions, hairpins, internal loops or bubbles, stacked pairs, multi-branch loops, bulge loops or bulges, pseudoknots, and junctions. As used herein, “duplexes” refers to regions of sequence complementarity that form stable, double-stranded structures following Watson-Crick base-pairing rules. “Hairpins” contain an uninterrupted sequence of unpaired bases flanked by two regions of complementarity that form a duplex or stem region. “Internal loops” contain a complementary duplexed region interrupted by two sequences of unpaired bases, one on each “strand.” “Bulges” are internal loops with a complementary duplexed region interrupted by a single sequence of unpaired bases on one strand. “Stacked pairs” are loops formed by pairing of i^{th} nucleotide with j^{th} and i+1^{th} nucleotide with j−1^{th}. “Multi-branch loops” of specific sequence are loops with more than two strands with unpaired sequences. “External bases” are free bases not contained in a loop. “RNA pseudoknots” are tertiary structural elements that result when a loop in a secondary structure pairs with a complementary sequence outside the loop. See, e.g., Chastain, M. and Tinoco Jr., I., Prog. Nucleic Acid Res. Mol. Biol. 41:131-177 (1991); Pleij in Gesteland and Atkins, THE RNA WORLD (Cold Spring Harbor Laboratory Press, Cold Spring, N.Y. 1993).

As used herein, a “peptidomimetic” is a synthetic compound that is structurally similar to a peptide, but contains non-peptidic structural elements. A peptidomimetic lacks at least one classical peptide characteristic, such as enzymatically scissile peptidic bonds. For example, a peptidomimetic can have an unnatural backbone, e.g., oligocarbamate (with a carbamate-containing backbone); oligourea (with a urea-containing backbone), e.g., as described in Tamilarasu et al., Bioorg. Med. Chem. Lett. 10(9):971-974 (2000); Tamilarasu et al. J. Am. Chem. Soc. 121:1597-1598 (1999); oligothiourea (with a thiourea-containing backbone); and N-substituted peptoids (peptidomimetics that results from the oligomeric assembly of N-substituted amino acids), e.g., oligopeptoid ester or amide analogs, e.g., as described in Kesavan et al., Bioconjug. Chem. 13(6):1171-1175 (2002).

The new methods provide several advantages. As one example, the methods described herein can be used to rapidly identify small molecule therapeutic leads for targeting specific RNA-protein interactions. The methods are also sufficiently sensitive to be used to screen a library of very similar compounds, all of which bind to the same target RNA, to determine which bind with the greatest affinity, avidity, and/or specificity, and to calculate binding constants including IC₅₀. Nanomolar concentrations of target RNA and test compounds can be detected with the new methods, alleviating the need for large amounts of starting material.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic illustration of the sequence and secondary structure of trans-activation responsive (TAR) RNA used in structural studies.

FIGS. 2A and 2B are line graphs illustrating fluorescence anisotropy data for β-peptides 1 and 2 binding to fluorescein-tagged wild-type TAR (FIG. 2A) and bulge-deleted TAR (FIG. 2B). Each data point represents five observations; curve fittings were performed as described herein. Black triangles, β-peptide 1, gray circles, β-peptide 2.

### DETAILED DESCRIPTION

The present invention relates to high throughput fluorescence polarization/anisotropy (FPA) assay screening methods of sufficient sensitivity and specificity to identify small molecules that bind to preselected target RNAs and specifically disrupt RNA-protein interactions. The methods can be used to screen combinatorial libraries, e.g., small molecule libraries. The compounds to be screened can be free in solution, e.g., in a well of a multi-well plate, or can be attached to a solid support, e.g., a support that is capable of containing an aqueous environment, e.g., a surface, e.g., the bottom of a multi-well plate, or a support that is capable of being suspended in an aqueous environment, e.g., beads.

Thus, the present invention includes methods for screening combinatorial libraries for agents capable of specifically disrupting RNA-protein interactions. The methods described herein are performed under conditions that are optimized to increase the specificity and sensitivity of the assay to allow the detection of the difference in mobility of fluor-labeled target RNA molecules caused by specific binding of small molecules. In part, the increased specificity and sensitivity of the assay are achieved by (1) using small target RNA molecules of about 10-100, typically 10-50 or 10-30 nucleotides in length, and (2) placement of the fluorescent dye molecules close to the putative binding pocket in the target RNA, but not so close as to interfere with binding. Typically, the fluorochrome is placed about 5-10 bases away, and/or is attached to modified U or C nucleosides, sugars, or the backbone of the target RNA.

Increasing the difference in the anisotropy value between free RNA and the bound RNA-test compound complex is an additional factor in the high sensitivity of the present methods. Fluorescence anisotropy reveals the average angular displacement of the fluorophore that occurs between absorption and subsequent emission of a photon. This angular displacement is dependent upon the rate and extent of rotational diffusion during the lifetime of the excited state. In turn, these diffusive motions depend on the size and shape of the rotating molecule. The present methods include optimized conditions and parameters, using short RNA sequences of defined structure, and small molecule test compounds to achieve a significant increase in anisotropy.

Screening of Small Molecule Test Compounds for Binding to a Target RNA

The invention includes methods for using target RNAs to screen small molecule test compounds or libraries of compounds to identify test compounds that bind to the target RNA in a fluorescence polarization/anisotropy (FPA) assay. In some embodiments, the target RNA is labeled, e.g., fluor-labeled. In other embodiments, the small molecules are labeled, e.g., fluor-labeled. In some embodiments, screening includes contacting a target RNA, e.g., a fluor-labeled target RNA, with a test compound in solution (e.g., both the target RNA and test compound are free-floating in the solution). In some embodiments, screening includes contacting a target RNA, e.g., a fluor-labeled target RNA, in solution with a solid support to which a test compound is attached. In other embodiments, screening includes contacting a target RNA, e.g., a fluor-labeled target RNA, attached to a solid support with a test compound in solution. Typically, the contacting occurs in an aqueous solution. The aqueous solution typically stabilizes the target RNA and prevents RNA denaturation or degradation without interfering with binding of the test compounds. In some embodiments, the aqueous solution is similar to the solution in which a complex between the target RNA and its natural ligand is formed in vitro. For example, TK buffer (20 mM Tris-HCl, pH 8.0/50 mM KCl), which is commonly used to form protein-RNA complexes in vitro, including Tat-TAR RNA, Rev-RRE, human capping enzymes-mRNA, and human P-TEFb-7SK RNA complexes, can be used in the methods described herein as an aqueous solution to screen a library of test compounds for RNA binding compounds.

The methods for screening a test compound attached to a solid support can include contacting a test compound with a target RNA in the presence of an aqueous solution, e.g., an aqueous solution including a buffer and a combination of salts.

The aqueous solution can also, in addition or instead, include unlabeled target RNA having a mutation at a known or suspected protein binding site, which renders the unlabeled RNA incapable of interacting with a test compound at that site. For example, if dye-labeled TAR RNA is used to screen a library, unlabeled TAR RNA having a mutation in the uracil 23/cytosine 24 bulge region (“bulge-deleted TAR RNA”) can also be present in the aqueous solution. Without being bound by any theory, the addition of unlabeled RNA that is essentially identical to the dye-labeled target RNA except for a mutation at the protein binding site minimizes interactions of other regions of the dye-labeled target RNA with test compounds or with the solid support, thus minimizing non-specific interactions and preventing false positive results.

The aqueous solution can also include a protein binding partner (or a relevant portion thereof) of a target RNA molecule, where one is known. The relevant portion of the protein binding partner of the target RNA can be a peptide that includes the minimal sequence necessary for binding to the target RNA. Methods for determining the minimal required sequence are known in the art. In this way, the IC₅₀ of a small molecule test compound for inhibiting the protein-target RNA interaction can be measured, e.g., by measuring the binding of the target RNA to its binding partner in the absence and presence of varying concentrations of the test compound. As one example, where the target RNA is derived from the TAR RNA sequence, the aqueous solution can also include a peptide derived from the relevant portion of Tat, e.g., Tat amino acids 47-57.

The solution typically further includes a buffer, a combination of salts, and optionally, a detergent or a surfactant. The pH of the solution typically ranges from about 5 to about 9, e.g., from about 6 to about 8, or from about 6.5 to about 7.5. A variety of buffers may be used to achieve the desired pH. Suitable buffers include, but are not limited to, Tris, Mes, Bis-Tris, Ada, Aces, Pipes, Mopso, Bis-Tris propane, Bes, Mops, Tes, Hepes, Dipso, Mobs, Tapso, Trizma, Heppso, Popso, TEA, Epps, Tricine, Gly-Gly, Bicine, and sodium-potassium phosphate. The solution typically includes from about 10 mM to about 100 mM, preferably from about 25 mM to about 75 mM, most preferably from about 40 mM to about 60 mM buffer. The pH of the aqueous solution can be optimized for different screening reactions, depending on the target RNA used and the type of test compound used, and therefore, the type and amount of the buffer used in the solution can vary from screen to screen. In one embodiment, the aqueous solution has a pH of about 7.4, which can be achieved, e.g., using about 50 mM Tris buffer.

In addition to an appropriate buffer, the aqueous solution typically also includes a combination of salts, from about 2 mM to about 20 mM KCl, from about 1 mM to about 100 mM NaCl, and from about 0 mM to about 20 mM MgCl₂. Without being bound by any theory, it is believed that a combination of KCl, NaCl, and MgCl₂ may stabilize the target RNA such that most of the RNA is not denatured or digested over the course of the screening reaction. The optional concentration of each salt used in the aqueous solution is dependent on the particular target RNA used and can be determined using routine experimentation.

In some embodiments, the aqueous solution can further include from about 0.01% to about 0.5% (w/v) of a detergent or a surfactant. Without being bound by any theory, a small amount of detergent or surfactant in the solution might reduce non-specific binding of the target RNA to the solid support and control aggregation and increase stability of target RNA molecules. Typical detergents useful in the present methods include, but are not limited to, anionic detergents, such as salts of deoxycholic acid, 1-heptanesulfonic acid, N-laurylsarcosine, lauryl sulfate, 1-octane sulfonic acid and taurocholic acid; cationic detergents such as benzalkonium chloride, cetylpyridinium, methylbenzethonium chloride, and decamethonium bromide; zwitterionic detergents such as CHAPS, CHAPSO, alkyl betaines, alkyl amidoalkyl betaines, N-dodecyl-N,N-dimethyl-3-ammonio-1-propanesulfonate, and phosphatidylcholine; and non-ionic detergents such as n-decyl α-D-glucopyranoside, n-decyl β-D-maltopyranoside, n-dodecyl β-D-maltoside, n-octyl β-D-glucopyranoside, sorbitan esters, n-tetradecyl β-D-maltoside, and tritons. Typically, the detergent, if present, is a nonionic detergent. Typical surfactants useful in the methods described herein include, but are not limited to, ammonium lauryl sulfate, polyethylene glycols, butyl glucoside, decyl glucoside, Polysorbate 80, lauric acid, myristic acid, palmitic acid, potassium palmitate, undecanoic acid, lauryl betaine, and lauryl alcohol. In some embodiments, the detergent, if present, is Triton X-100 and is present in an amount of about 0.1% (w/v).

In some embodiments, non-specific binding of a dye-labeled target RNA to a test compound or to the solid support can be further minimized by pre-treating the test compound or combinatorial library with one or more blocking agents. Thus, in some embodiments, the test compound or combinatorial library is treated with a blocking agent, e.g., bovine serum albumin (hereinafter “BSA”), before contacting with to the dye-labeled target RNA. In another embodiment, the test compound or combinatorial library is treated sequentially with at least two different blocking agents. In some embodiments, the test compound or combinatorial library is contacted with in a solution including BSA and is washed with a BSA-free solution to remove unbound BSA. This blocking step can be performed, e.g., at 4°-37° C., e.g., at room temperature, for about 0.5 to about 3 hours, e.g., about 0.5 to 1 hour. In a subsequent step, the test compound or library can optionally be contacted with a solution including unlabeled RNA having a mutation at the putative or known protein binding site, e.g., a mutation predicted or known to disrupt secondary structure. This blocking step is typically performed at about 4°-37° C., e.g., at room temperature, for about 0.5 hours to about 1 hour. Finally, the test compound or library is typically washed in an RNA-free solution to remove unbound, unlabeled RNA. Typically, the solution used in the one or more blocking steps is substantially similar to the aqueous solution used to screen the library with the dye-labeled target RNA, e.g., is similar in pH and salt concentration.

Once contacted, the mixture of dye-labeled target RNA and the test compound is typically maintained at 4°-37° C., e.g., at room temperature, for a sufficient amount of time to allow binding of test compounds to target RNA, e.g., for about 1 minute to about 1 hour, e.g., about 5 minutes to about 30 minutes, e.g., with constant agitation, e.g., stirring. Small molecules that bind to the target RNA are then identified using FPA (as described in further detail below); small molecules that show an increase in fluorescence polarization are binding to the target RNA. To identify other test compounds in the library that bind less tightly to the target RNA, the solid support can subsequently be exposed to additional dye-labeled target RNA for additional time, e.g., from about 1 hour to about 3 hours or longer, and additional positive areas identified.

Small molecules that show an increase in fluorescence polarization, indicating that the molecules are binding to the target RNA, can be evaluated further for their specificity for particular nucleotides within the target RNA. This can be accomplished by systematically mutating the nucleotides within a region of secondary structure of the target RNA (e.g., for TAR RNA, nucleotides that represent the regions that confer specificity for Tat binding, nucleotides in the bulge, e.g., nucleotides 22-26, 39, and 40, and the loop, e.g., nucleotides 30-35). Using the FPA assay to evaluate changes in binding affinity to the mutated RNA sequences will reveal the nucleotides important for a particular small molecule to bind the target RNA.

Fluorescence Polarization/Anisotropy (FPA) Assay

Briefly, an FPA assay involves exciting a fluorescent molecule using polarized light and evaluating the loss of polarization of the emitted light. The FPA can then be correlated with the mobility of the fluorescent molecule. When a fluorescent molecule absorbs polarized light, it can become depolarized provided the molecule can rotate in solution to a significant degree (representing its mobility) over the finite period that the fluorescent molecule emits light. Typically, using a fluor-labeled ligand with a low molecular weight, a significant amount of depolarization can occur over the course of the lifetime of the fluorescent emission and a low polarization signal can be achieved. If the fluor-labeled smaller molecule binds to a much larger molecule, the amount of molecular rotation of the smaller molecule occurring during the fluorescence lifetime is significantly reduced because of the added weight of the larger molecules. Thus, the depolarization signal becomes slight and the polarization signal becomes large. See, e.g., Royer et al., U.S. Pat. Nos. 5,445,935, 5,765,292, and 6,326,142.

FPA assays for binding studies involving fluor-labeled RNA as described herein are successful in part because the time scale of the rotation of RNA is comparable to the decay time of the fluorescence of molecules such as fluorescein (or other dyes, e.g., commercially available dyes) that can be conjugated to RNA. It is theorized that when a small molecule binds to a fluorescein-labeled RNA, tumbling of the fluorescein in solution dramatically decreases. This decrease in tumbling due to the binding of the small molecule to the RNA, results in an increase in the polarization signal. According to this principle, the binding affinity can be quantitatively determined from either polarization or anisotropy measurements.

In an FPA assay, two separate intensities are measured for each data point through polarizers installed in both excitation and emission light paths. For one measurement, the polarizers are oriented parallel to each other (I_{II}); the other measurement is acquired with the polarizer oriented perpendicular to each other (I_{⊥}). Fluorescence polarization is described in terms of polarization (P) defined as equation 1:

$$P = \frac{{I}_{II} - {I}_{⊥}}{{I}_{II} + {I}_{⊥}} \qquad ( 1 )$$

A measurement of anisotropy (r) is defined as equation 2:

$$r = \frac{{I}_{II} - {I}_{⊥}}{{I}_{II} + 2 {I}_{⊥}} \qquad ( 2 )$$

Anisotropy is a dimensionless quantity, which is independent of the total intensity of the sample.

A number of devices are known in the art and commercially available for use in measuring FPA; for example, SAPPHIRE™ (Tecan) or VICTOR™ series (Perkin Elmer). Each machine can be optimized to increase the sensitivity of detection, depending on the experimental parameters, such as the excitation and emission spectra of the fluorochrome used to label the target RNA. For example, the slit width, wavelength of excitation light and emission detection, G-factor, and lamp voltage can be optimized to enhance sensitivity.

Target RNA Molecules

The target RNA molecule can be any sequence. The RNA can be derived from any source, including, but not limited to, animals (e.g., mammals such as humans), plants (e.g., food crops such as rice or corn), bacteria (e.g., pathogenic bacteria), and viruses (e.g., HIV or HCV). In some embodiments, the target RNA molecule is derived from, e.g., has the sequence of a portion or a region of, a coding RNA sequence (e.g., an mRNA) or a non-coding RNA sequence (e.g., rRNA, snRNA, hnRNA, snoRNA, mRNA, RNase P RNA, telomerase RNA, 4.5S RNA, 7SL RNA, tmRNA, hY RNA, RNase MRP RNA, tRNA and/or a regulatory RNA) that is regulated by a protein-RNA or RNA-RNA interaction (e.g., by splicing, capping, adenylation, or other RNA processing, e.g., to affect cellular localization, translation, RNA export, and/or RNA stability, e.g., half-life) or regulates a protein by a protein-RNA or RNA-RNA interaction (e.g., by regulating transcription, post-transcription, or protein function). A number of suitable non-coding, nontranslated RNAs are known in the art, e.g., as described in Szymanski et al., J. Appl. Genet. 44(1):1-19 (2003), Lease and Belfort, Proc. Natl. Acad. Sci. USA 97:9919-9924 (2000), and Altuvia and Wagner, Proc. Natl. Acad. Sci. USA 97(18):9824-9826 (2000), inter alia. Methods for identifying suitable target RNA sequences are known in the art, e.g., as described in Wang and Rana, Methods in Molecular Biology, 118:49-62 (1999); Sassetti et al., Proc Natl Acad Sci USA. 98(22):12712-7 (2001); Sassetti and Rubin, Curr Opin Microbiol 5:27-32 (2002); Johansson and Cossart, Trends Microbiol. 11(6):280-5 (2003).

A number of suitable sources of target RNA sequences are listed in U.S. Pat. No. 6,503,713, incorporated herein by reference in its entirety.

The target RNA can be, e.g., derived from an RNA in a pathogen, e.g., bacteria, e.g., Escherichia coli, Mycobacterium tuberculosis (tuberculosis), Neisseria gonorrhoeae, Neisseria meningitidis (meningococcus), Rickettsiae, Coxiella burnetii, Salmonellae, e.g., Salmonella enterica or Salmonella typhi, Staphylococcus aureus, Streptococcus pyogenes, or Treponema pallidum; viruses, e.g., cytomegalovirus, hantaviruses, hepatitis, e.g., Hepatitis A, B, C or D, herpes simplex virus, Human Immunodeficiency Virus (HIV), Molluscum contagiosum, papillomavirus, or SARS (Severe Acute Respiratory Syndrome) virus; infectious fungi, e.g., Pneumocystis jerovici; or parasites, e.g., giardia lamblia, roundworms, scabies, or tapeworms. For example, a bacterial rRNA can be used as a source of target RNA, to screen for a small molecule useful in the treatment of subjects infected with the bacteria, e.g., drug resistant bacterial strains. Other bacterial RNA can also be used, e.g., mRNA or noncoding RNA as described herein. Preferably the target RNA is derived from an RNA that is essential for the viability of the pathogen, so that targeting the pathogen RNA is useful in treating conditions associated with the pathogen.

The target RNA molecule can be, e.g., about 10-100 nucleotides (nt) long, e.g., about 10-75 nt long, about 10-50 nt long, about 10-30 nt long, about 15-35 nt long, about 35-50 nt long, or about 50-75 nt long.

The target RNA molecule will typically contain at least one region of known or predicted secondary structure. Various regions of secondary structure are known in the art, and can be derived from, e.g., structure-activity studies or crystallographic structural studies. Regions of predicted secondary structure can be identified using methods known in the art, e.g., manually or using computational methods. A number of programs are known in the art for predicting secondary structure. Such predictive computer modeling programs include, but are not limited to the following: Mfold (Zuker et al. in RNA Biochemistry and Biotechnology, 11-43, Barciszewski and Clark, eds., NATO ASI Series, Kluwer Academic Publishers, (1999)); RNAstructure (Mathews et al., Journal of Molecular Biology 288:911-940 (1999)), RNAfold in the Vienna RNA Package (Hofacker et al. 17, A-1090 Wien, Austria), Tinoco plot (Tinoco et al., Nature 230, 363-367 (1971)), ConStruct, which seeks conserved secondary structures (Steger and Riesner, J. Mol. Biol. 258, 813-826 (1996); and Luck et al. Nucleic Acids Res. 21:4208-4217 (1999)), FOLDALIGN, (Gorodkin et al., Nucleic Acids Res. 25(18):3724-3732, (1997); and Gorodkin et al., ISMB 5:120-123 (1997)), and RNAdraw (Matzura and Wennborg, Computer Applications in the Biosciences (CABIOS), 12(3) 247-249 (1996)).

Other suitable target RNA molecules with specific secondary structures can be found in databases such as the Small RNA Database (Perumal et al., Department of Pharmacology, Baylor College of Medicine, USA), Database of non-coding RNAs (Erdman et al., Nucleic Acids Res. 29: 189-193 (2001)), large subunit rRNA database (Wuyts et al., Nucleic Acids Res. 29(1): 175-177 (2001)), the small subunit rRNA database (Wuyts et al., Nucleic Acids Res. 30,183-185 (2002)), snoRNA Database for budding yeast (Lowe and Eddy, Science 283: 1168-1171 (1999)), for Archaea (Omer et al., Science 288: 517-522 (2000)), for Arabidopsis thaliana (Brown et al., RNA 7: 1817-1832 (2001)), tRNA sequences and sequences of tRNA genes (Sprinzl and Gauss, Nucleic Acids Res. 12(Suppl):r1-r57 (1984); Sprinzl et al., Nucl. Acids Res. 26:148-153 (1998); Lowe and Eddy, Nucl. Acids Res. 25, 955-964 (1997); Transfer-RNA: Structure, properties and recognition, Schimmmel et al., Eds., Cold Spring Harbor Laboratory, p. 518-519 (1979); Steinberg and Kisselev, Biochimie 74:337-351 (1992); Limbach et al., Nucl. Acids Res. 22, 2183-2196 (1994); Crain and McCloskey, Nucl. Acids Res. 25:126-127 (1997) Sprinzl et al., on the world wide web at uni-bayreuth.de/departments/biochemie/trna/), the 5S ribosomal RNA database (Szymanski et al., Nucleic Acid Res. 30: 176-178 (2002)), The Nucleic Acid Database Project (NDB) at Rutgers University (on the world wide web at ndbserver.rutgers.edu/NDB/), or The RNA Structure Database (on the world wide web at RNABase.org)). If a portion of an RNA sequence is known to be involved in a protein-RNA interaction, that portion can be selected for use as a target RNA molecule.

Target RNA can be synthesized using methods known in the art, e.g., enzymatically and/or chemically synthesized, e.g., using phosphoramidite or other solution or solid-phase methods, or in vitro transcription. Detailed descriptions of the chemistry used to form polynucleotides by the phosphoramidite method are well known (see, e.g., Caruthers et al., U.S. Pat. Nos. 4,458,066 and 4,415,732; Caruthers et al., Genetic Engineering 4:1-17 (1982); Users Manual for Models 392 and 394 Polynucleotide Synthesizers, 1990, pages 6-1 through 6-22, Applied Biosystems, Part No. 901237). The phosphoramidite method of polynucleotide synthesis is typically used because of its efficient and rapid coupling and the stability of the starting materials. The synthesis is typically performed with the growing polynucleotide chain attached to a solid support, such that excess reagents, which are generally found in the liquid phase, can be easily removed by washing, decanting, and/or filtration, thereby eliminating the need for purification steps between synthesis cycles.

The target RNAs are typically labeled at one or more specific locations. Fluorescent labels can be incorporated into or attached to the target RNA during or after synthesis using methods known in the art. A label should not be incorporated into a target RNA at a site at which test compounds are likely to bind (e.g., in a site likely to be in or near the secondary structure), since the presence of a covalently attached label might interfere sterically or chemically with the binding of the test compounds at this site. Accordingly, if the region of the target RNA that binds to an in vivo ligand is known, a detectable label is preferably incorporated into the RNA molecule at one or more positions that are spatially or sequentially remote from the binding region. Typically, the label is incorporated about 3-20 bases away, e.g., about 5-15 bases away, or about 5-10 bases away from the binding site. In some embodiments, the fluorochrome can be attached to a target RNA molecule with nucleoside modification at C and/or U at the C5 position using an appropriate linker (e.g., a linker 2-12 carbons long (Shah et al., Bioconjugate Chemistry, 5:508-512 (1994). In other examples, sugar residues can be fluor-labeled at the 2 prime position, e.g., using 2 prime amino or thio groups, which are commercially available. In addition or alternatively, the fluorochrome can be incorporated using a backbone modification, e.g., using a phosphorothioate-containing RNA, also commercially available. As one example, a trans-activation responsive (TAR)-derived target RNA as described herein can be labeled with a fluorophore that is attached near the bulge or loop sequence by using modified U or C nucleoside, sugars, or backbone.

Suitable labels include, but are not limited to, fluorescein or other fluorochromes, e.g., commercially available dyes such as eosin, BODIPY™, Alexa Fluor™, fluorescein, Oregon Green, tetramethylrhodamine, quantum dots, umbelliferone, fluorescein isothiocyanate, rhodamine, dichlorotriazinylamine fluorescein, dansyl chloride, phycoerythrin and Texas Red.

After synthesis, the labeled target RNA can be purified using standard techniques known to those skilled in the art (see, e.g., Hwang et al. Proc. Natl. Acad. Sci. U.S.A. 96(23):12,997-13,002 (1999) and references cited therein). Depending on the length of the target RNA and the method of its synthesis, purification techniques can include, but are not limited to, reverse-phase high-performance liquid chromatography (“reverse-phase HPLC”), fast performance liquid chromatography (“FPLC”), and gel purification. After purification, the target RNA is refolded into its native conformation, typically by heating to approximately 85-95° C. and slowly cooling to room temperature in a buffer, e.g., a buffer including about 50 mM Tris-HCl, pH 8 and 100 mM NaCl.

For example, Tat-TAR interactions provide an ideal model system for developing drugs that target specific RNA structures, useful in the treatment of HIV.

Small Molecules and Combinatorial Libraries

The invention includes methods for high throughput screening of small molecule test compounds, e.g., compounds that are initially members of an organic chemical library, to identify agents that specifically bind a target RNA. As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries (Pergamon-Elsevier Science Limited, 1998), and include those such as the “split and pool” or “parallel” synthesis techniques, solid-phase and solution-phase techniques, and encoding techniques (see, for example, Czarnik, Curr. Opin. Chem. Bio. 1(1):1-2, 60-66 (1997). In addition, a number of small molecule libraries are commercially available. A number of suitable small molecule test compounds are listed in U.S. Pat. No. 6,503,713, incorporated herein by reference in its entirety

Libraries screened using the methods of the present invention can include a variety of types of small molecule test compounds. A given library can include a set of structurally related or unrelated small molecule test compounds. In some embodiments, the test compounds are peptide or peptidomimetic molecules. In some embodiments, test compounds include, but are not limited to, peptide analogs including peptides comprising non-naturally occurring amino acids, e.g., β-amino acids or α-substituted α-amino acids (“β³-amino acids”), phosphorous analogs of amino acids, such as α-amino phosphonic acids and α-amino phosphinic acids, or amino acids having non-peptide linkages, or other small organic molecules.

The small molecules can include one or more peptidomimetics, peptoids, tripeptides, tetrapeptides, pentapeptides, hexapeptides, heptapeptides, octapeptides, nonapeptides, decapeptides, or larger, e.g., 20-mers or more. In some embodiments, the small molecules include cyclic peptides. In some embodiments, the small molecules include β-peptides, e.g., β-peptides based on non-β peptide small molecules such as tetrapeptides, pentapeptides, hexapeptides, heptapeptides, octapeptides, nonapeptides, or decapeptides. In some embodiments, all of the small molecules are β-peptides. In some embodiments, the small molecules include peptides having at least one non-natural amino acid, e.g., a D-amino acid. In some embodiments, each of the plurality of small molecules is a peptide containing at least one non-natural amino acid. In some embodiments, the plurality of small molecules includes at least one peptidomimetic containing at least one beta amino acid residue; in other embodiments, each of the plurality of small molecules is a peptidomimetic containing at least one beta amino acid residue. In some embodiments, the plurality of small molecules includes at least one peptidomimetic containing at least one beta peptide backbone. In some embodiments, each of the plurality of small molecules is a peptidomimetic containing at least one beta amino acid peptide backbone.

In some embodiments, the plurality of small molecules includes heterocyclic compounds containing at least one of: a carbon atom, a nitrogen atom, an oxygen atom, a sulfur atom, a benzene ring, a cyclohexane ring, and a cyclopentane ring. The small molecules containing heterocyclic structures can also include a charged residue with a net positive charge, or a charged residue with a net negative charge.

The small molecule combinatorial libraries useful in the methods described herein can include the types of compounds that will potentially bind to the ligand binding sites of the target RNAs used to screen them. For example, where the target RNA has a known protein binding partner, the test compounds can be structurally similar to the known binding partner. As one example, if TAR RNA is used to screen a library for test compounds that bind to the TAR RNA Tat binding site, the test compounds can be peptides or peptidomimetics that are structurally similar to the natural Tat peptide that binds TAR RNA with high affinity.

In some embodiments, the small organic molecules and libraries thereof can be obtained by systematically altering the structure of a first small molecule, e.g., a first small molecule that is structurally similar to a known natural binding partner of the target RNA, or a first small molecule identified as capable of binding the target RNA, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

Small molecules identified as “hits” (e.g., small molecules that demonstrate an increase in fluorescence polarization signal) in the first screen are selected and systematically altered, e.g., using rational design, to optimize binding affinity, avidity, specificity, or other parameter. Such optimization can also be screened for using the methods described herein. Thus, in one embodiment, the invention includes screening a first library of small molecules using the methods described herein, identifying one or more hits in that library, subjecting those hits to systematic structural alteration to create a second libraries of compounds structurally related to the hit, and screening the second library using the methods described herein. In another embodiment, the invention includes screening a first library of small molecules using a non-FPA method known in the art, identifying one or more hits in that library, subjecting those hits to systematic structural alteration to create a second library of compounds structurally related to the hit, and screening the second library using the methods described herein.

Small molecules identified as hits can be considered candidate therapeutic compounds, useful in treating disorders associated with the specific interaction between the source of the target RNA sequence and its protein binding partner. Such disorders include inflammatory disorders; viral diseases, e.g., Hepatitis C virus (HCV), Human Immunodeficiency Virus (HIV) and other infections; and other diseases involving gene expression, e.g., neoplasms and disorders associated with abnormal or unwanted cellular differentiation or proliferation, e.g., cancers. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disease, e.g., cancer, inflammation, or a viral infection. One skilled in the art will also recognize that these techniques can also be used to monitor the synthesis of test compounds.

The present invention also includes small inorganic or organic molecules, e.g., β-peptide molecules; peptidomimetics (e.g., peptoid oligomers, e.g., peptoid amide or ester analogues, D-peptides, L-peptides, oligourea or oligocarbamate); small peptides (e.g., tripeptides, tetrapeptides, pentapeptides, or larger); cyclic peptides; α-amino acids or β-substituted β-amino acids (“β³-amino acids”), phosphorous analogs of amino acids, such as α-amino phosphonic acids and α-amino phosphinic acids, or amino acids having non-peptide linkages, or other small organic molecules, e.g., other non-natural or unnatural peptide-like structures; and inorganic molecules (e.g., heterocyclic ring molecules) that have been identified using the methods described herein (e.g., identified as hits).

β-Peptides

In some embodiments, the small molecules are β-peptides. β-peptides are non-biological polymers synthesized from beta amino acids. The study of β-peptides has accelerated over the past decade, propelled by demonstrations that they can be programmed to adopt protein-like secondary structures. These structures have given rise to a variety of biological activities, and the protease resistance of β-peptides makes them attractive from a pharmaceutical standpoint (see, e.g., Cheng et al., Chem. Rev. 101:3219-3232 (2001); Gademann et al., Curr. Med. Chem. 6:905-925 (1999); Werder et al., Helv. Chim. Acta 82:1774-1783 (1999); Gademann et al., Int. Ed. Engl., 38:1223-1226 (1999); Hamuro et al., J. Am. Chem. Soc. 121:12200-12201 (1999); Porter et al., Nature 404:565 (2000); Seebach et al., Chimia 52:734-739 (1998); and Seebach et al., Chimia 55:345-353 (2001), incorporated herein by reference in their entirety).

As demonstrated herein (see Example 1, below), β-peptide 1 displays an enhanced specificity for wild-type TAR RNA as compared to a wild-type α-peptide 3. Although the affinity of β-peptide 1 for wild-type TAR is roughly 15-fold lower than that of the homologous α-peptide 3, the affinity of β-peptide 1 for bulgeless TAR is reduced by more than two orders of magnitude compared to α-peptide 3. Thus, in one embodiment, the invention includes a method for enhancing the specificity of an RNA-targeting compound comprising an α-peptide, by converting it to a β-peptide.

In some embodiments, the method includes screening libraries comprising β-peptide combinatorial libraries based on small molecules such as trimer, tetramer, pentamer, hexamer, heptamer, octamer, nonamer, or decamer (or higher) structures, e.g., β-peptide analogs of trimers, tetramers, pentamers, hexamers, heptamers, octamers, nonamers, or decamers or other small molecules, e.g., small molecules as described in Hwang et al., J. Biol. Chem., 10.1074/jbc.M301749200 (2003).

A Model System: Tat-TAR Protein-RNA Interaction

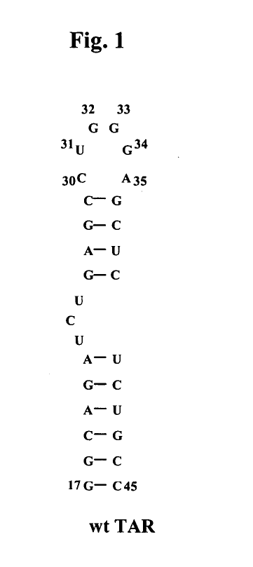

Tat-TAR protein-RNA interactions are essential elements in the mechanism of HIV-1 gene expression, and provide a useful model for protein-RNA interactions in general, as well as potential therapeutic targets for the treatment, prevention, or delay of HIV-1 infection. Tat-TAR interactions provide an ideal model system for developing drugs that target specific viral RNA structures, useful in the treatment of HIV. HIV-1 is a complex retrovirus that encodes six regulatory proteins including Tat, which is essential for viral replication. Tat is an RNA-binding protein that requires specific interactions with RNA structures called TAR (trans-activation responsive) for increasing the rate of HIV-1 replication during the HIV-1 life cycle. Tat protein acts by binding to the TAR RNA element, a 59-nucleotide (nt) stem-loop structure located at the beginning of all newly made HIV-1 transcripts. TAR RNA contains a 6-nt loop and a 3-nt pyrimidine bulge that separates two helical stem regions (FIG. 1). TAR RNA spans the minimal sequences that are required for Tat responsiveness in vivo (Jakobovits et al., Mol. Cell. Biol. 8:2555-2561(1988) and for in vitro binding of Tat-derived peptides (Cordingley et al., Proc. Natl. Acad. Sci. 87:8985-8989 (1990). Wild-type TAR contains two non-wild-type base pairs to increase transcription by T7 RNA polymerase (Wang et al., J. Biol. Chem. 271:16995-16998 (1996); see FIG. 1). The loop and bulge regions are important for mediating Tat-TAR interactions. Mutational analysis of HIV-1 Tat protein has identified two important functional domains: an arginine-rich region that is required for binding to TAR RNA, and an activation domain that mediates its interactions with the cellular machinery also required for HIV-1 replication.

The search for new therapies for AIDS has focused on events in the life cycle of HIV other than reverse transcription and proteolysis. Forward transcription of viral genomic RNA from proviral DNA is an attractive target, particularly because inhibition of transcription might prevent reactivation of latent or suppressed HIV infection. Transcription of HIV RNA requires the interaction of the virally encoded Tat protein with the transcriptional activator-responsive element (TAR), a bulged RNA hairpin structure formed by the nascent transcript. The key determinants of the Tat-TAR interaction have been localized to a trinucleotide bulge in TAR RNA and the 11-amino acid basic region of Tat (residues 47-57; YGRKKRRQRRR, SEQ ID NO:1). This interaction can be disrupted by a variety of backbone-modified Tat 47-57 analogues, including a D-peptide (Huq et al., Biochemistry 38:5172-5177 (1999), an oligocarbamate (Tamilarasu et al., Bioorg. Med. Chem. 11:505-507 (2001), an oligourea (Tamilarasu et al. (2001) supra), and various peptoid-based structures (e.g., Kesavan et al., Bioconj. Chem. 13(6):1171-1175 (2002).

The affinity of the Tat basic sequence for TAR RNA depends on two key features: a single arginine side chain, which may specifically bridge two phosphates in the TAR bulge, and a cluster of cationic residues, which appears to provide a polyelectrolyte-like affinity for RNA. Backbone-modified Tat analogues preserve the side chains of the Tat basic region, but vary (in spacing or chirality) the relative positions of the functional groups. The success of these analogues in disrupting the Tat-TAR interaction provides further evidence that the presentation of an arginine side chain in a cationic context is a primary determinant of affinity. Tat analogs, or fragments such as the Tat 47-57 peptide, which comprise at least a minimal portion of the TAR RNA binding site, can be used in conjunction with a target RNA based on the TAR RNA sequence, e.g., as shown in FIG. 1, which includes the relevant portion of the TAR sequence, e.g., the portion or portions determined to be involved in the Tat-TAR binding interaction. See, e.g., Examples 1 and 4.

Therapeutic Compounds

As described herein, small molecules identified as hits can be considered candidate therapeutic agents, which are potentially useful in treating, delaying, or preventing the development or progression of disorders associated with the specific interaction between the source of the target RNA sequence and its protein binding partner. The candidate compounds may be effective as is, or can be optimized, e.g., modified, e.g., derivatized, to become therapeutic agents. One of skill in the art will appreciate that the disease or diseases which the therapeutic agent will be useful in treating will depend on the nature of the target RNA, and the nature of the target-RNA protein interaction that is disrupted by the small molecule. For example, these therapeutic agents can be administered to a patient, e.g., a mammal, typically a human, to prevent or treat a disease, e.g., cancer, inflammation, or a viral infection.

“Treatment” or “treating” refers to an amelioration of cancer, inflammation, or a viral infection, or at least one discernible symptom thereof, e.g., an amelioration of at least one measurable physical parameter, not necessarily discernible by the patient. Typically, “treatment” or “treating” refers to inhibiting the progression of cancer, inflammation, or a viral infection, either physically, e.g., stabilization of a discernible symptom, physiologically, e.g., stabilization of a physical parameter, or both. “Delay” or “delaying” refers to delaying the onset of cancer, inflammation, or a viral infection. As used herein, “prevention” or “preventing” refers to a reduction of the risk of acquiring cancer, inflammation, or a viral infection.

Thus, in one embodiment, a compound identified as a hit using a method described herein is administered as a preventative measure to a patient. According to this embodiment, the patient can have a genetic or a non-genetic predisposition to cancer, inflammation, or a viral infection. Accordingly, the compounds described herein can be used for the treatment of one manifestation of cancer, inflammation, or a viral infection and/or prevention of a manifestation of cancer, inflammation, or viral infection.

The compounds may be useful for treating or preventing a variety of cancers, including, but not limited to, leukemias, including but not limited to acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, Lymphomas including but not limited to Hodgkin's disease, non-Hodgkin's disease, Multiple mycloma, Waldenstrom's macroglobulinemia, Heavy chain disease, Solid tumors including but not limited to sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

The compounds may be useful for treating or preventing several types of inflammation, including, but not limited to, eczema, inflammatory bowel disease, rheumatoid arthritis, asthma, psoriasis, ischemia/reperfusion injury, ulcerative colitis and acute respiratory distress syndrome.

The compounds may be useful for treating or preventing a variety of viral infections, including, but not limited to those caused by infection with hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In some embodiments, the therapeutic compound is a β-peptide. In some embodiments, the therapeutic compound is β-peptide 1, and it is useful in the treatment of HIV infection.

Methods of Administration

Due to their activity, the compounds identified as hits by the methods described herein are useful in veterinary and human medicine, e.g., to treat, prevent or delay the development of cancer, inflammation, or a viral infection in a patient.

When administered to a patient, a compound described herein is typically administered incorporated into a pharmaceutical composition, e.g., compositions that optionally comprise a pharmaceutically acceptable vehicle. Thus, the invention also includes pharmaceutical compositions including one or more compounds identified as hits by the methods described herein. The pharmaceutical compositions can be administered by any suitable and convenient route, for example, orally, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the compounds described herein. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a compound described herein into the bloodstream.

In some embodiments, it may be desirable to administer a compound locally. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In some embodiments, it may be desirable to introduce a compound described herein into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In some embodiments, the compounds described herein can be formulated as a suppository, with traditional binders and vehicles such as triglycerides.

In some embodiments, the compounds identified as hits can be delivered in a vesicle, e.g., a liposome (see Langer, Science 249:1527-1533 (1990); Liposomes in the Therapy of Infectious Disease and Cancer, Lopez-Berestein and Fidler (eds.), (Liss, N.Y., (1989).

In yet another embodiment, the compounds described herein can be delivered in a controlled release system (see, e.g., Goodson, in Medical Applications of Controlled Release, supra, vol. 2, pp. 115-138 (1984)). Other controlled-release systems discussed in the review by Langer, Science 249:1527-1533 (1990) may be used. In some embodiments, a pump may be used (see Langer, supra; Sefton, CRC Crit. Ref. Biomed. Eng. 14:201 (1987); Buchwald et al., Surgery 88:507 (1980) Saudek et al., N. Engl. J. Med. 321:574 (1989). In other embodiments, polymeric materials can be used (see, e.g., Medical Applications of Controlled Release, Langer and Wise (eds.), (CRC Pres., Boca Raton, Fla. 1974); Controlled Drug Bioavailability, Drug Product Design and Performance, Smolen and Ball (eds.), (Wiley, N.Y. 1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989). In yet another embodiment, a controlled-release system can be placed in proximity of a target of a compound described herein, e.g., a particular RNA, thus requiring only a fraction of the systemic dose.

The pharmaceutical compositions can optionally comprise a suitable amount of a pharmaceutically acceptable vehicle so as to provide the form for proper administration to the patient.

Pharmaceutically acceptable vehicles can be vehicles approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a compound described herein is administered. Suitable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the pharmaceutically acceptable vehicles are typically sterile. Water can be used as a vehicle, e.g., when the compound described herein is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The pharmaceutical compositions, if desired, can also contain minor amounts of wetting or emulsifying agents, or pH buffering agents.

The pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., U.S. Pat. No. 5,698,155). Other examples of suitable pharmaceutical vehicles are described in Remington's Pharmaceutical Sciences, Alfonso R. Gennaro ed., Mack Publishing Co. Easton, Pa., 19th ed., 1995, pp. 1447 to 1676.

In some embodiments, the compounds are formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. Typically, compositions for intravenous administration comprise sterile isotonic aqueous buffer. Where necessary, the compositions may also include a solubilizing agent.

In another embodiment, the compounds described herein can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compounds described herein are to be administered by infusion, they can be dispensed, for example, with an infusion bottle containing sterile pharmaceutical grade water or saline. Where the compounds described herein are administered by injection, an ampoule of sterile water for injection or saline can be provided so that the ingredients may be mixed prior to administration.

The amount of a compound described herein that will be effective in the treatment, prevention or delay of development of a particular type of cancer, inflammation, or viral infection disclosed herein will depend on the nature of the compound and the nature of the disorder or condition, and can be determined by standard clinical techniques. In addition, in vitro or in vivo assays can be employed to help identify optimal dosage ranges. The precise dose to be employed will also depend on the route of administration, and the seriousness of the disease or disorder, and should be decided according to the judgment of the practitioner and each patient's circumstances. Suitable dosage ranges for oral administration can be about 0.001 milligram to about 200 milligrams of a compound described herein or a pharmaceutically acceptable salt thereof per kilogram body weight per day. In some embodiments, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound described herein is administered, or if a compound described herein is administered with a therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound described herein per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses can be extrapolated from dose-response curves derived from in vitro or animal model test systems. Such animal models and systems are well known in the art.

The invention also provides pharmaceutical packs or kits comprising one or more vessels containing a compound described herein. Optionally associated with such container(s) can be a notice in the form prescribed by a governmental agency regulating the manufacture, use or sale of pharmaceuticals or biological products, which notice reflects approval by the agency of manufacture, use or sale for human administration. In some embodiments, the kit contains more than one compound described herein. In some embodiments, the kit comprises a therapeutic agent and a compound described herein.

The compounds are typically assayed in vitro and in vivo, for the desired therapeutic or prophylactic activity, prior to use in humans. For example, in vitro assays can be used to determine whether it is preferable to administer a compound described herein alone or in combination with another compound described herein and/or a therapeutic agent. Animal model systems can be used to demonstrate safety and efficacy.

Other methods will be known to the skilled artisan and are within the scope of the invention.

Combination Therapy

In certain embodiments of the present invention, a compound described herein can be used in combination therapy with at least one other additional therapeutic agent. The compound described herein and the additional therapeutic agent can act additively or, more preferably, synergistically. In some embodiments, a composition comprising a compound described herein is administered concurrently with the administration of an additional therapeutic agent, which can be part of the same composition as or in a different composition from that comprising the compound described herein. In some embodiments, a composition comprising a compound described herein is administered prior or subsequent to administration of an additional therapeutic agent. As many of the disorders that the compounds are useful in treating are chronic, in one embodiment, combination therapy involves alternating between administering a composition comprising a compound described herein and a composition comprising an additional therapeutic agent, e.g., to minimize the toxicity associated with a particular drug. The duration of administration of the compound described herein and/or additional therapeutic agent can be, e.g., one month, three months, six months, a year, or for more extended periods. In some embodiments, when a compound described herein is administered concurrently with an additional therapeutic agent that potentially produces adverse side effects including, but not limited to, toxicity, the additional therapeutic agent can advantageously be administered at a dose that falls below the threshold at which the adverse side is elicited.

The additional therapeutic agent can be an anti-cancer agent. Useful anti-cancer agents include, but are not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, γ-radiation, alkylating agents including nitrogen mustard such as cyclophosphamide, Ifosfamide, trofosfamide, Chlorambucil, nitrosoureas such as carmustine (BCNU), and Lomustine (CCNU), alkylsulphonates such as busulfan, and Treosulfan, triazenes such as Dacarbazine, platinum containing compounds such as Cisplatin and carboplatin, plant alkaloids including vinca alkaloids, vincristine, Vinblastine, Vindesine, and Vinorelbine, taxoids including paclitaxel, and Docetaxol, DNA topoisomerase inhibitors including Epipodophyllins such as etoposide, Teniposide, Topotecan, 9-aminocamptothecin, campto irinotecan, and crisnatol, mytomycins such as mytomycin C, and Mytomycin C, anti-metabolites, including anti-folates such as DHFR inhibitors, methotrexate and Trimetrexate, IMP dehydrogenase inhibitors including mycophenolic acid, Tiazofurin, Ribavirin, EICAR, Ribonuclotide reductase Inhibitors such as hydroxyurea, deferoxamine, pyrimidine analogs including uracil analogs 5-Fluorouracil, Floxuridine, Doxifluridine, and Ratitrexed, cytosine analogs such as cytarabine (ara C), cytosine arabinoside, and fludarabine, purine analogs such as mercaptopurine, thioguanine, hormonal therapies including receptor antagonists, the anti-estrogens Tamoxifen, Raloxifene and mcgestrol, LHRH agonists such as goscrclin, and Leuprolide acetate, anti-androgens such as flutamide, and bicalutamide, retinoids/deltoids, Vitamin D3 analogs including EB 1089, CB 1093, and KH 1060, photodyamic therapies including vertoporfin (BPD-MA), Phthalocyanine, photosensitizer Pc4, Demethoxy-hypocrellin A, (2BA-2-DMHA), cytokines including Interferon-α, Interferon-γ, tumor necrosis factor, as well as other compounds having anti-tumor activity including Isoprenylation inhibitors such as Lovastatin, Dopaminergic neurotoxins such as 1-methyl-4-phenylpyridinium ion, Cell cycle inhibitors such as staurosporine, Actinomycins such as Actinomycin D and Dactinomycin, Bleomycins such as bleomycin A2, Bleomycin B2, and Peplomycin, anthracyclines such as daunorubicin, Doxorubicin (adriamycin), Idarubicin, Epirubicin, Pirarubicin, Zorubicin, and Mitoxantrone, MDR inhibitors including verapamil, and Ca.sup.2+ ATPase inhibitors such as thapsigargin.

The additional therapeutic agent can be an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, colchicine, allopurinol, probenecid, sulfinpyrazone and benzbromarone.

The additional therapeutic agent can be an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

High-Throughput Screening of Small Molecule Libraries

For high throughput screens, plates, e.g., plates with 96, 384, or more separate areas separated by a barrier, e.g., wells can easily be screened. Suitable plates are known in the art, and can be manufactured, modified, or are commercially available. In some embodiments, each area, e.g., each well, contains a unique small molecule of known or unknown structure, or a pool of molecules of known or unknown structure. The compounds to be screened can be free in solution, e.g., in a well of a multi-well plate, or can be attached to a solid support, e.g., a support that is capable of containing an aqueous environment, e.g., a surface of a multi-well plate, e.g., the bottom, or to a solid support that is or can be suspended is solution, e.g., beads. The target RNA can be free in solution, e.g., in a well of a multi-well plate, or can be attached to a solid support that is capable of providing an aqueous environment, e.g., a surface of a multi-well plate, e.g., the bottom. Thus, in some embodiments, the small molecule and the target RNA are both free in solution. In other embodiments, either the small molecule or the target RNA molecule is attached to a solid support, e.g., a distinct area of the solid support, e.g., the bottom of a well of a multi-well plate, e.g., using a flexible linker known in the art, e.g., a 3-40 carbon diethylene linker. In some embodiments, coated solid supports, e.g., solid supports coated with streptavidin can be used, e.g., with biotin-linked target RNA or test compounds. In some embodiments, beads, e.g., TetaGel® Resin, are used as solid support. TentaGel® resin has good swelling properties in a broad range of organic solvents as well as in aqueous media. Because of the long polyethyleneglycol spacer on TentaGel® beads, ligands on the beads behave kinetically as though they were in solution. Thus in some embodiments, the combinatorial library is synthesized using TentaGel® resin as a solid support, and the target RNA, e.g., labeled target RNA, is incubated in a suspension of library beads. A number of suitable solid supports are known in the art, and many are commercially available.

In some embodiments, the library or a subset thereof is contained in a plurality of wells of a multi-well plate; e.g., with each well containing one or more unique small molecules that is different from the small molecules in at least one of the other wells. In some embodiments, the multi-well plate also includes one or more positive and/or negative control wells. Negative control wells can contain, for example, no small molecules, unlabeled target RNA molecules, mutated target RNA molecules with disrupted secondary structure, or other negative control. Positive control wells can contain, for example, known binding partners of the target RNA, e.g., a nucleic acid molecule that is complementary to the target RNA, and large enough to affect an increase in anisotropy or fluorescence polarization. In some embodiments, a number of multi-well plates, each comprising a unique set of small molecules, are screened. In this way, a library of small molecules in the millions can be screened for identification of RNA-targeting small molecules.

### EXAMPLES

The invention is further described in the following examples, which do not limit the scope of the invention described in the claims.

### Example 1

### Fluorescence Polarization Assay Method

Using the methods described herein, it has been demonstrated that a α-amino acid oligomer (“β-peptide”) analogue of Tat 47-57 (“β-peptide 1”) binds TAR RNA with nanomolar affinity.

β-Peptide and mutant β-peptide binding affinities to TAR RNA were measured by fluorescence anisotropy method on a PTI (Photon Technology International) fluorescence spectrophotometer. A T-format setup based on two emission channels was used. The intensities of the parallel and perpendicular components simultaneously were obtained in one measurement using two separate detectors. The excitation side is a motorized Glan-Thompson polarizer and emission sides are two film polarizers placed in vertical and horizontal orientation, respectively. The anisotropy can be calculated from equation 1,

$$r = \frac{{I}_{II} - {I}_{⊥}}{{I}_{II} + 2 {I}_{⊥}} \qquad ( 1 )$$

where I_{II} and I_{⊥} are fluorescence intensities observed when the emission polarizer is oriented parallel and perpendicular to the direction of the polarized excitation, respectively.

The fluorescein-labeled TAR RNA (F-TAR, either wt TAR or bulge-less TAR, see FIG. 1) was excited at 490 nm and two polarized emissions were monitored at 512 nm. The slits were set at 8 nm for both excitation and emission light. The integration time was 20 seconds. All data were measured in a Sub-Micro cuvette (Starna Cell, Inc.) with a starting volume of 160 μL. Each addition of β-peptide/mutant β-peptide was followed by one-minute equilibration before the fluorescence signal was recorded. All experiments were done at room temperature and in a TKT buffer (TKT: 50 mM Tris-HCl, pH=7.4, 20 mM KCl and 0.02% Tween20). The initial concentration of F-TAR was around 25 nM. The stock concentration of β-peptide/mutant β-peptide for titration was 2 μM. The experiment was repeated three times independently. For every single point, five measurements were made and their average anisotropy value was used for calculation of the K_{D}.

The binding dissociation constant (K_{D}) is determined by fitting the plot of anisotropy of F-TAR versus the concentration of the β-peptide using equation 3,

$$A = \frac{- ( {K}_{D} + C + Cap ) + \sqrt{{( {K}_{D} + C + Cap )}^{2} - 4 \cdot C \cdot Cap}}{2} \cdot a \qquad ( 3 )$$

where A is the relative fluorescence anisotropy of dye-labeled TAR RNA. The capacity for binding, Cap, is the maximum binding ability of a small molecule. C is the total concentration of β-peptide added. a is a correction factor.

The affinity of Tat protein and Tat-derived peptides for TAR is known to depend sensitively on assay conditions. For example, electrophoresis-derived K_{d} values for extremely similar peptides can differ by two orders of magnitude with varying salt concentrations (70 mM NaCl vs. 20 mM KCl) (Wang et al., J. Am. Chem. Soc. 119:6444-6445 (1997); Calnan et al., Genes Dev. 5:201-210 (1991)). Therefore, the difference in K_{d} values for α-peptide 3 between our assay and one reported previously is not completely unexpected. The K_{d} and binding mode of α-peptide 3 to fluorescein-tagged TAR have been verified by FRET to rhodamine-tagged α-peptide 3.

Because of this variation in measured K_{d} values, a standard procedure in this field is to report dissociation constant ratios (Kᵣₑₗ), in which a value above 1.0 denotes stronger binding of the analogue relative to the native peptide. The Kᵣₑₗ value for β-peptide 1 relative to α-peptide 3 is 0.072, indicating a significant diminution of affinity when the backbone is altered to a peptide backbone. Interestingly, alteration of the backbone to an oligocarbamate gives Kᵣₑₗ=0.69 (Wang et al., J. Am. Chem. Soc. 119:6444-6445 (1997)), and alteration to an oligourea gives Kᵣₑₗ=7.1, (Tamilarasu et al., J. Am. Chem. Soc. 121:1597-1598 (1999)) indicating that affinity of Tat analogues for TAR cannot be explained as a simple function of side chain spacing.

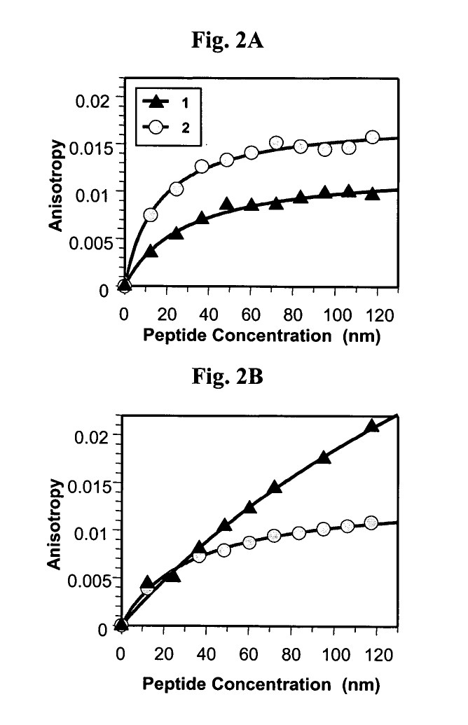

The results are shown in Table 1 (see wild type) and FIGS. 2A-B. FIG. 2A shows the fluorescence anisotropy data for β-peptides 1 and 2 binding to fluorescein-tagged wild-type TAR RNA. FIG. 2B shows the fluorescence anisotropy data for β-peptides 1 and 2 binding to fluorescein-tagged bulge-deleted TAR RNA

TABLE 1
Dissociation constants for β-peptides 1 and 2, and
from wild-type or bulge-deleted (mut) TAR RNA as
determined by FPA. Assays were performed in 20 mM KCl

| | |
|---|---|
| Kd/nM | Binding Specificity |

| | | |
|---|---|---|
| wt TAR | mut TAR | (Kd (mutTAR)/Kd (wtTAR)) |

| | | | |
|---|---|---|---|
| wt β-peptide 1 | 28 ± 4 | 281 ± 68 | 10 |
| mut β-peptide 2 | 16 ± 2 | 33 ± 2 | 2 |
| α-TatR 3 | 1.8 ± 0.5 | 1.1 ± 0.2 | 1 |
| α-TatK 4 | 32 ± 4 | 72 ± 8 | 2 |

α-TatR = Tat 47-57 sequence, i.e., N-Tyr-Gly-Arg-Lys-Lys-Arg-Arg-Gln-Arg-Arg-Arg (SEQ ID NO: 1); Alpha-TatK = Tat 47-57 where all Arg are replaced with Lys.

The binding of β-peptide 1 to labeled wild-type or mutant bulge-deleted target TAR RNA increased the molecular weight of the fluorescein-bearing complex, decreasing the mobility of the fluorescent molecule and, hence, increasing polarization. Thus, under the optimized conditions described herein, small molecules such as the β-peptide 1 used here cause a large enough decrease in mobility of the target RNA to be detectable using FPA.

Furthermore, the β-peptide 1 specifically binds to the TAR RNA bulge range. When a bulge-less TAR RNA was used instead of a wild type TAR RNA, the binding affinity of β-peptide 1 to mutant TAR RNA decreases about 10 fold, indicating that the assay methods described herein can detect specific binding.

Surprisingly, the intended “negative control” mutant β-peptide 2 binds more tightly to TAR in this assay than does either designed β-peptide 1 or “negative control” α-peptide 4 (α-TatK) the affinity of which for TAR has not previously been reported. The mutant beta peptide 2 binds with high affinity, but less specificity to the target TAR RNA. The alpha peptides 3 and 4 show little specificity, binding to either the mutant or wild type RNA. It is difficult, however, to attribute these differences to specific binding of the TAR bulge by an “arginine fork” motif, because similar trends are seen in the affinities of peptides 2-4 for the bulgeless control RNA. The lack of specificity of an α-peptide of Tat 49-57 for wild-type TAR over bulgeless TAR has been previously reported (Kamine et al., Virology 182:570-577 (1991)).

It is therefore even more surprising to find that β-peptide 1 displays an enhanced specificity for wild-type TAR RNA as compared to α-peptide 3. Because the bulgeless hairpin is a common secondary structural motif in RNA, specificity for the bulged stem-loop is a requirement for any effective therapeutic directed at TAR. Although the affinity of β-peptide 1 for wild-type TAR is roughly 15-fold lower than that of α-peptide 3, the affinity of β-peptide 1 for bulgeless TAR is reduced by more than two orders of magnitude compared to α-peptide 3. Appending a minimum of 8 random amino acids to Tat 49-57 has been reported to increase specificity, (Kamine et al., supra) but the increased length of β-peptide 1 (11 atoms, less than the length of 4 α-amino acids) cannot be solely responsible for the increased specificity. Similarly, including the 10-residue Tat “helical core region” N-terminal to the basic region gives a modest increase in peptide specificity for wild-type TAR over mutants (Kamine et al., supra; Churcher et al., J. Mol. Biol. 230:90-110 (1993)), but this effect may require four residues at the N-terminus of the core (Wang et al., Biochemistry 40:6458-6464 (2001)). Thus, the methods described herein are useful for detecting specific binding. In addition, β-peptide small molecules, such as β-peptide 1 described herein, can bind target RNA with greater specificity than natural α-peptides. Thus, the specificity of an α-peptide small molecule binding agent may be enhanced by the modification of one or more residues to β-amino acids, and/or the inclusion of one or more β-peptide backbones.

### Example 2

### Synthesis of β-Peptides

The syntheses of β-peptide 1 (as previously reported, Umezawa et al., J. Am. Chem. Soc. 2002, 124, 368-369) and β-peptide 2, in which all arginine side chains have been replaced by lysine side chains (YGKKKKKQKKK; SEQ ID NO:2), were carried out by automated solid-phase methods from Fluorenylmethyloxycarbonyl (Fmoc)-protected β-substituted β-amino acids (“β³-amino acids”) obtained enantiospecifically using Müller's modification of Seebach's methodology (see, e.g., Seebach et al., Chimia 55 345-353 (2001), incorporated herein by reference in its entirety). Fmoc-β³HArg(Pmc) obtained by homologation, as previously reported (Umezawa, supra), requires repeated column chromatography, with fraction-by-fraction HPLC analysis, to eliminate small (≦1%) amounts of starting material Fmoc-Arg(Pmc) impurity. α-Lys impurity was also detected in Fmoc-β³HLys(Boc) and its oligomers, albeit at lower levels. If chromatography is performed only once, in highly redundant sequences these impurities give rise to a population of α-Arg- and α-Lys-containing contaminants, detectable by mass spectrometry but inseparable from the desired β-peptide by HPLC. Peptides used for the studies described herein were synthesized using Fmoc-β³HArg(Pmc) and Fmoc-β³HLys(Boc) containing undetectable (<0.05%) amounts of Fmoc-α-amino acid.

### Example 3

### Combinatorial Library of TAR-Derived Target RNA Binding Agents

A combinatorial library to be screened for agents that would specifically disrupt the Tat-TAR binding interaction was developed. Based on previous screening results and to create charged residues, 14 D-amino acids, and Lys and Arg were included. As one theory, it is believed that the charge (electrostatic) interactions between ligands and the phosphodiester backbone of RNA play an important role in TAR recognition, therefore, a number of other charged side chains were introduced in the library design. Moreover, the charged ligands are exposed in the aqueous media and therefore more accessible to the protein receptors.

Previous studies showed that Tat-derived carbamate biopolymer binds TAR RNA with high affinity and specificity. The library also included carbamate monomers to further explore the relationship between the structural changes on the peptide backbone and the binding specificity to target molecules. Peptoid monomers were introduced to remove intramolecular hydrogen bonds (C═O . . . H—N) and to alter the steric hindrance of side chains in the peptide backbone. As one theory, the peptoid and carbamate bonds are expected to be more stable to enzymatic degradation. The aromatic monomers were chosen to cover the potential stacking and hydrophobic interactions in the target molecule. Aminobenzoic acid monomers were included to test the effect of sp2 backbone geometry and to gain rigid conformational structure. Also included were sulfonyl, halogenated, aminophenyl, nitrophenylalanine, aromatic and cyclic secondary amines, γ-substituted glutamic acids, enantiopure α-substituted prolines, and β-turn mimetics to cover a variety of functional groups as well as to generate structures of rigid scaffolds in the library.

### Example 4

### High Throughput Screening of a 96-Well Microplate

As a test experiment for screening the binding affinity of small molecules to TAR RNA, FP label technology was applied in a VICTOR²V® plate reader system. The Wallace 1420 VICTOR²V® (Perkin-Elmer) is a multilabel, multitask plate reader that can measure prompt fluorescence, FP, luminescence, time-resolved fluorescence (TRF) and absorbance, and can handle both fast and slow kinetic measurements by using up to 4 injectors.

Two filters were selected for these experiments, F485 for excitation and F535 for emission. The slits were set to 1-10 nm for both excitation and emission lights, typically 4 mm. The excitation lamp control was set to constant voltage and G-factor was set to unity. The measurement time was one second per well. The binding assay of fluorescein-labeled TAR-binding ligand (e.g., Tat 47-57 alpha or beta) to wild type unlabeled TAR RNA was performed in a 96-well microplate. Each individual well contained 100 μL of 10 nM ligand in TK buffer (20 mM KCl, 50 mM Tris, pH 7.4). Different amounts of TAR RNA (0-2 μL, 1 μM or 10 μM) were added into the wells. Only free peptide was included in wells H-11 and H-12 as control. The polarization (mP unit) was recorded in a VICTOR²V plate reader at room temperature.

The results were illustrated using a pseudocolor scale, with a blue color representing low mP values and red color representing high mP values. The polarization of free peptide was around 180 mP and a value of 590 mP obtained for the tightest binding.

In an alternative method, RNA can be labeled with the dye and unlabeled ligands can be used to identify the interactions. To probe interactions at different sites, the dye can be incorporated at internal sequence position, at the backbone, or at the terminal positions.

These results demonstrate that the FPA assay methods described herein are sufficiently sensitive and specific to be successfully used as a high-throughput screen. Thus, these FP assays can be applied to screen heterocyclic and small molecule libraries to identify RNA binding compounds.

### Example 5

### Screening a Combinatorial Library for Target RNA Binding Agents Using FPA

A combinatorial library as described in Example 3 is prepared for screening by suspending each test compound in an aqueous solution and placing each test compound into at least one well of one or more 384-well plate. Also included in the plate are wells containing compounds suitable for use as positive and negative controls: Tat 47-57 peptide (either alpha or beta) as a positive control, and an empty well without any test compound as a negative control. One or more wells including unlabeled, mutant target RNA as a negative controls are also included. Fluor-labeled TAR RNA is added to each well (one or more wells are omitted as additional negative controls). The plate is then screened substantially as described in Example 4.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of simultaneously screening a plurality of test compounds for their ability to bind to a target RNA molecule, the method comprising:
incubating a plurality of test compounds with a target RNA molecule comprising a fluorescent label under conditions that enable the binding of the test compounds to the target RNA molecule; and
assaying fluorescence polarization of the target RNA molecule;
wherein an increase in fluorescence polarization of the target RNA molecule as compared to a level of fluorescence polarization of the target RNA molecule in the absence of any test compounds indicates that one of the test compounds binds to the target RNA molecule.

2. The method of claim 1, wherein the plurality of test compounds comprises at least five test compounds.

3. The method of claim 1, wherein the incubating is in a housing comprising a plurality of individual areas, each area comprising a target RNA and one or more of the plurality of test compounds.

4. The method of claim 3, wherein the housing is a multiwell plate.

5. The method of claim 1, wherein the target RNA molecule comprises at least one secondary structure.

6. The method of claim 5, wherein the secondary structure is selected from the group consisting of a hairpin, an internal loop, a stacked pair, a multi-branch loop, an external base, and a bulge.

7. The method of claim 1, wherein the test compounds are small molecules.

8. The method of claim 7, wherein the small molecules comprise one or more of a peptidomimetic, peptoid, tripeptide, tetrapeptide, pentapeptide, hexapeptide, heptapeptide, octapeptide, nonapeptide, or decapeptide.

9. The method of claim 7, wherein at least one of the small molecules comprises a cyclic peptide.

10. The method of claim 7, wherein at least one of the small molecules comprises a peptide.

11. The method of claim 10, wherein the β-peptide corresponds to a non-β peptide small molecule.

12. The method of claim 11, wherein the non-β-peptide small molecule is selected from the group consisting of tetrapeptides, pentapeptides, hexapeptides, heptapeptides, octapeptides, nonapeptides, and decapeptides.

13. The method of claim 1, wherein the test compounds comprise peptides each having at least one non-natural amino acid.

14. The method of claim 13, wherein the non-natural amino acid is a D-amino acid.

15. The method of claim 1, wherein the target RNA molecule is less than or equal to about 100 bases long.

16. The method of claim 1, wherein the fluorescent label is selected from the group consisting of fluorescein, eosin, dibenzopyrrometheneboron difluoride dyes, fluorescein, Oregon Green, tetramethylrhodamine and Texas Red.

17. The method of claim 1, wherein the target RNA molecule is derived from microRNA (mRNA).

18. The method of claim 1, wherein the target RNA molecule is derived from mRNA.

19. The method of claim 1, wherein the target RNA molecule is derived from nuclear RNA.

20. The method of claim 1, wherein the target RNA molecule is derived from ribosomal RNA.

21. The method of claim 1, wherein the target RNA molecule is derived from regulatory RNA.

22. A method of simultaneously screening a plurality of test compounds to identify a candidate compound for the treatment of a pathogen-associated condition, the method comprising:
incubating a plurality of test compounds with a target RNA molecule derived from the pathogen, wherein the target RNA comprises a fluorescent label, under conditions that enable the binding of the test compound to the target RNA molecule; and
assaying fluorescence polarization of the target RNA molecule;
wherein an increase in the fluorescence polarization of the target RNA molecule as compared to a level of fluorescence polarization of the target RNA molecule in the absence of any test compounds indicates that one of the test compounds is a candidate compound for the treatment of the pathogen-associated condition.

23. The method of claim 22, wherein the pathogen-associated condition is a viral infection and the target RNA molecule is derived from viral RNA.

24. The method of claim 23, wherein the viral RNA is selected from the group consisting of Human Immunodeficiency Virus (HIV), Hepatitis C Virus (HCV), and Severe Acute Respiratory Syndrome (SARS).

25. The method of claim 24, wherein the HIV RNA is selected from the group consisting of trans-activation response element (TAR) and Rev response element (RRE).

26. The method of claim 22, wherein the pathogen-associated condition is a bacterial infection and the target RNA molecule is derived from RNA of the bacterium associated with the condition.

27. The method of claim 26, wherein the target RNA molecule is derived from bacterial rRNA.

28. The method of claim 26, wherein the bacterial RNA is from a bacterium selected from the group consisting of Escherichia coli, Mycobacterium tuberculosis, Neisseria gonorrhoeae, Neisseria meningitidis, Rickettsiae, Coxiella burnetii, Salmonellae, Staphylococcus aureus, Streptococcus pyogenes, or Treponema pallidum.

29. The method of claim 22, further comprising administering a candidate compound to an animal model of the pathogen-associated condition, wherein an improvement in a symptom of the animal model indicates that the candidate compound is a candidate therapeutic compound for the treatment of the pathogen-associated condition.

30. The method of claim 29, further comprising administering the candidate therapeutic compound to a subject having the pathogen-associated condition, wherein an improvement in the subject indicates that the compound is a therapeutic agent.

31. The method of claim 30, wherein the subject is a human in a clinical trial.

32. The method of claim 22, wherein the pathogen-associated condition is a bacterial infection, and the method further comprises contacting a candidate compound with the bacterium associated with the condition, and evaluating an effect of the candidate compound on the viability of the bacterium, wherein a reduction in the viability of the bacterium indicates that the compound is a candidate therapeutic compound for the treatment of the bacterial infection.
